(19) United States
(12) Patent Application Publication
(10) Pub. No.: US 2024/0324874 A1
(43) Pub. Date: Oct. 3, 2024

(54) TECHNIQUES TO GENERATE EXTERNAL LIMBUS-BASED CORNEOSCLERAL TOPOGRAPHY

(71) Applicant: Oregon Health & Science University, Portland, OR (US)

(72) Inventors: David Huang, Portland, OR (US); Yan Li, Portland, OR (US); Clara Llorens Quintana, Portland, OR (US)

(73) Assignee: Oregon Health & Science University, Portland, OR (US)

(21) Appl. No.: 18/622,638

(22) Filed: Mar. 29, 2024

Related U.S. Application Data
(60) Provisional application No. 63/493,674, filed on Mar. 31, 2023.

(51) Int. Cl. A61B 3/10; A61B 3/00

(52) U.S. Cl. CPC A61B 3/102; A61B 3/0025; A61B 3/1005

(57) ABSTRACT

Various embodiments herein provide methods to obtain corneoscleral surface profiles from optical coherence tomography (OCT) data and/or another three-dimensional (3D) volumetric scan of the eye. The OCT data may be obtained using radial scans. The limbal junction may be estimated from the radial profiles of the central moments of OCT axial scans. The external topographic limbus may be located using curve fitting of a scleral topography map and/or corneal topography map. Elevation profiles and topographic maps of the anterior eye, referenced to the best-fit topographic limbal circle and its associated plane and central axis, are constructed. The reference coordinate allows the generation of maps and radial profiles for surface elevation, axial radius, and tangential radius. Using ellipsoidal fitting, the corneal height, corneal principal meridians and associated radii of curvature, and scleral principal meridians and associated radii of curvature, are calculated.

### CROSS-REFERENCE TO RELATED APPLICATION

The present application claims priority to U.S. Provisional Patent Application No. 63/493,674, filed Mar. 31, 2023, the entire disclosure of which is hereby incorporated by reference.

### ACKNOWLEDGEMENT OF GOVERNMENT SUPPORT

This invention was made with government support under R01 EY028755 and R01 EY029023 awarded by the National Institutes of Health. The government has certain rights in the invention.

### TECHNICAL FIELD

The present disclosure generally relates to the field of ophthalmology. In particular, apparatuses, systems, and methods for external limbus-based corneoscleral topography.

### BACKGROUND

The limbus is the transition area between the cornea and the sclera in the eye. Demarcating the corneoscleral limbus is important for fitting scleral lenses. A scleral lens is a rigid contact lens that vaults over the cornea and the limbus, resting entirely on the conjunctival surface over the sclera. Scleral lenses are often fitted following a diagnostic approach in which an initial lens is chosen from a trial set and the lens parameters are refined by a trial-and-error process. The number of trials needed to find the best lens ranges between 1 and 8 lenses. For practitioners, scleral contact lens fitting is a long learning curve and the success of the fitting is highly dependent on their experience. Accurate measurement of corneoscleral topography, including the limbal size and shape, could enable a more objective and streamlined process for contact lens fitting and may allow more customized lens design.

Histologically, the external limbus is bound by the termination of the Bowman's layer and the beginning of the conjunctiva. The internal limbus is the bound by the termination of the corneal endothelium (Schwalbe's line) and the scleral spur. Topographically the corneoscleral limbus can be identified by the transition between the greater corneal radial curvature to the flatter sclera. When it comes to scleral lens fitting, the limbal transition between the steeper curvature of the cornea and the flatter curvature of the sclera determines appropriate diameter of the lens transition zone. The posterior lens surface should vault the limbus and the haptics should land on the perilimbal conjunctiva. Limbal measurements based nontopographical landmarks may not correspond with the topographical limbus and produce suboptimal scleral lens fitting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Embodiments will be readily understood by the following detailed description in conjunction with the accompanying drawings and the appended claims. Embodiments are illustrated by way of example and not by way of limitation in the figures of the accompanying drawings.

FIG. 1 depicts a schema of a scan pattern that includes 16 radial scans of 20 mm width centered at the pupil center, in accordance with various embodiments.

FIG. 2 illustrates a projection of the second central moment of the OCT axial scan signal along the transverse dimension, calculated from a radial cross section (B-frame). The limbus, as defined by the transition from the clear cornea to the white sclera, forms sharp transitions (e.g., as labeled) on this profile. The distance between the transitions corresponds to the moment-based limbus (e.g., as labeled). The moment drops sharply inside the iris edge (e.g., as labeled) and aids in locating this landmark.

FIG. 3 illustrates that external corneal surface inside the moment-based limbus is fitted with a 4th order polynomial (line 302) and the external conjunctival surface in the scleral region outside is fitted with a 2nd order polynomial (line 304). The external topographical limbus corresponds to the intersection of the two best-fit surfaces (dots labeled as “limbus”). The iris edges (labeled dots) mark the iris edges that defines the pupil.

FIG. 4 illustrates an en face projection of the topographic limbus measured on 16 meridians (dots 402), the best-fit ellipse (404), and its center (represented by diamond 406) in a left eye. The best-fit circle of the pupillary edge (dotted black line 408) and its center (cross) are also shown. The moment-based limbal ellipse (412, with center represented by diamond 414) is larger than the topographic limbus.

FIG. 5 illustrates an elevation topography of the corneal and scleral regions relative to the best-fit sphere (BFS), in accordance with various embodiments.

FIG. 6 illustrates an example process to generate corneoscleral topography, in accordance with various embodiments.

FIG. 7 is a flowchart of an example process to obtain the position of the external topographical limbus, in accordance with various embodiments.

FIG. 8 illustrates an example of manual internal limbus demarcation (e.g., mark 802), in accordance with various embodiments.

FIG. 9A illustrates an en face projection of the topographical limbus (dots 902) fitted with a circle (904). FIG. 9B illustrates the difference between the topographical limbus radial distance and the radius of the best-fit circle (dots 906) fitted with a 3-term Fourier model (line 908).

FIGS. 10A-10F illustrate double angle plots for ellipticity (FIGS. 10A and 10C) and toricity (FIGS. 10D and 10F) and triple angle plots for ovality (FIGS. 10B and 10E). The dots 1002 are the data points, the black square 1004 is the centroid of the data points, the inner line 1006 is the 95% confidence ellipse of the centroid and the outer line 1008 is the 95% confidence ellipse of the data points. The average total vector (centroid) magnitude and orientation is shown under each plot.

FIGS. 11A, 11B, and 11C illustrate correlation between the topographical limbal diameter and other types of limbal diameter measurements, including: internal limbus horizontal diameter (FIG. 11A), white-to-white (WTW) horizontal diameter (FIG. 11B), and internal limbus vertical diameter (FIG. 11C). The formulas for each linear regression are shown on the top of each plot together with the Pearson correlation coefficient R².

FIG. 12 schematically shows an example system for processing OCT datasets in accordance with the disclosure.

FIG. 13 schematically shows an example of a computing system in accordance with the disclosure.

### DETAILED DESCRIPTION OF DISCLOSED EMBODIMENTS

Various embodiments herein provide methods to generate extended-range-of-focus optical coherence tomography (OCT) radial scans and to obtain corneoscleral surface profiles. From each radial OCT scan, the limbal junctions and the pupil edges are first estimated from the radial profiles of the central moments of OCT axial scans. The conjunctival surface profile over the sclera outside the moment-based limbus was fit with a second-order polynomial curve. The corneal surface profile inside the moment-based limbus was fit with a fourth-order polynomial curve. The intersections of the two best-fit curves define the external topographic limbus. Combining radial profiles from meridians that span 360 degrees, the 3-dimensional topographic limbal boundary is generated. The conjunctival surface over the sclera outside the topographic limbus is fit with a Zernike polynomial series to obtain scleral topography. The corneal surface inside the topographic limbus is fit with a Zernike polynomial series to obtain corneal topography. Elevation profiles and topographic maps of the anterior eye, referenced to the best-fit topographic limbal circle and its associated plane and central axis, are constructed. The reference coordinate allows the generation of maps and radial profiles for surface elevation, axial radius, and tangential radius. Using ellipsoidal fitting, the corneal height, corneal principal meridians and associated radii of curvature, and scleral principal meridians and associated radii of curvature, are calculated.

The techniques described herein may enable locating the limbal junction between cornea and sclera using the radial profiles of the central moments of the OCT axial scans. Additionally, or alternatively, the embodiments herein may enable locating external topographic limbus using curve fitting of conjunctival surface in the scleral region and corneal surface in the central region. Furthermore, Zernike polynomial fitting of the conjunctival surface elevation map in the scleral region may be used to fill in any missing data. Additionally, embodiments may define a coordinate system reference to the best-fit topographic limbal circle to display corneoscleral topography and measure topographic parameters.

Accordingly, among other benefits, the embodiments described herein may be used to reliably identify the location of the limbal junction from OCT images without the need for segmentation. Additionally, embodiments may provide automatic detection of the external topographic limbus, and/or complete wide-field scleral topography. Furthermore, embodiments may provide a repeatable limbal map, corneoscleral topographic maps, and/or corneoscleral profiles to assist scleral lens fitting.

Anterior segment optical coherence tomography (AS-OCT) provides high-resolution cross-sectional images and has been used for the evaluation of the anterior corneoscleral profile and structures. Previous generations of anterior segment OCT instruments had limited ocular surface coverage and not enough speed to acquire the large number of scans needed for corneoscleral topography. This has only become possible recently with novel AS-OCT technologies that have increased speed and field of view.

A prior method locates the limbal junction by detecting abrupt changes in ocular surface curvature on radial OCT cross sections. Unfortunately, the local radius of curvature is very sensitive to small disturbances on the ocular surface such as pinguecula or scars. In addition, this method relies on the correct segmentation of the entire anterior ocular surface. Thus the previous method is prone to error.

In contrast, the method of limbus detection described herein, e.g., using central moment projection of the OCT axial scan signal, is robust; it does not require image segmentation and uses signal from the entire images. The method of topographic limbus location uses global shape fit and is minimally affected by small surface disturbances. Furthermore, the method provides complete 3-dimensional shape descriptions of cornea, limbus, and/or scleral that may be needed for customized fitting of a scleral contact lens.

In the present detailed description, reference is made to the accompanying drawings which form a part hereof, and in which are shown by way of illustration embodiments that can be practiced. It is to be understood that other embodiments can be utilized and structural or logical changes can be made without departing from the scope. Therefore, the following detailed description is not to be taken in a limiting sense, and the scope of embodiments is defined by the appended claims and their equivalents.

Various operations can be described as multiple discrete operations in turn, in a manner that can be helpful in understanding embodiments; however, the order of description should not be construed to imply that these operations are order dependent.

The description may use the terms “embodiment” or “embodiments,” which may each refer to one or more of the same or different embodiments. Furthermore, the terms “comprising,” “including,” “having,” and the like, as used with respect to embodiments, are synonymous.

In various embodiments, structure and/or flow information of a sample can be obtained using OCT (structure) and OCT angiography (flow) imaging based on the detection of spectral interference. Such imaging can be two-dimensional (2-D) or three-dimensional (3-D), depending on the application. Structural imaging can be of an extended depth range relative to prior methods, and flow imaging can be performed in real time. One or both of structural imaging and flow imaging as disclosed herein can be enlisted for producing 2-D or 3-D images.

Unless otherwise noted or explained, all technical and scientific terms used herein are used according to conventional usage and have the same meaning as commonly understood by one of ordinary skill in the art which the disclosure belongs. Although methods, systems, and apparatuses/materials similar or equivalent to those described herein can be used in the practice or testing of the present disclosure, suitable methods, systems, and apparatuses/materials are described below.

All publications, patent applications, patents, and other references mentioned herein are incorporated by reference in their entirety. In case of conflict, the present specification, including explanation of terms, will control. In addition, the methods, systems, apparatuses, materials, and examples are illustrative only and not intended to be limiting.

In order to facilitate review of the various embodiments of the disclosure, the following explanation of specific terms is provided:

A-scan: A reflectivity profile that contains information about spatial dimensions and location of structures with an item of interest (e.g., an axial depth scan). An A-scan is an axial scan directed along the optical axis of the OCT device and penetrates the sample being imaged. The A-scan encodes reflectivity information (for example, signal intensity) as a function of the depth of the sample being imaged. The A-scan encodes reflectivity information (for example, signal intensity) as a function of depth.

B-scan: A cross-sectional tomograph that can be achieved by laterally combining a series of axial depth scans (e.g., A-scans). A B-scan encodes planar cross-sectional information from the sample and is typically presented as an image. Thus, a B-scan can be called a cross sectional image.

Dataset: As used herein, a dataset is an ordered-array representation of stored data values that encodes relative spatial location in row-column-depth (x-y-z axes) format. In the context of OCT, as used herein, a dataset can be conceptualized as a three dimensional array of voxels, each voxel having an associated value (for example, an intensity value or a decorrelation value). An A-scan corresponds to a set of collinear voxels along the depth (z-axis) direction of the dataset; a B-scan is made up of set of adjacent A-scans combined in the row or column (x- or y-axis) directions. Such a B-scan can also be referred to as an image, and its constituent voxels referred to as pixels. A collection of adjacent B-scans can be combined to form a 3D volumetric set of voxel data referred to as a 3D image. In the system and methods described herein, the dataset obtained by an OCT scanning device is termed a “structural OCT” dataset whose values can, for example, be complex numbers carrying intensity and phase information. This structural OCT dataset can be used to calculate a corresponding dataset termed an “OCT angiography” dataset of decorrelation values reflecting flow within the imaged sample. There is a direct correspondence between the voxels of the structural OCT dataset and the OCT angiography dataset. Thus, values from the datasets can be “overlaid” to present composite images of structure and flow (e.g., tissue microstructure and blood flow) or otherwise combined or compared.

En Face angiogram/image: OCT angiography data can be presented as a projection of the three dimensional dataset onto a single planar image called an en face angiogram (Wallis J et al, Med Imaging IEEE Trans 8, 297-230 (1989); Wang R K et al, 2007 supra; Jia Y et al, 2012 supra; incorporated by reference herein). Construction of such an en face angiogram requires the specification of the upper and lower depth extents that enclose the region of interest within the retina OCT scan to be projected onto the angiogram image. These upper and lower depth extents can be specified as the boundaries between different layers of the retina (e.g., the voxels between the inner limiting membrane and outer plexiform layer can be used to generate a 2D en face angiogram of the inner retina). Once generated, the en face angiogram image can be used to quantify various features of the retinal vasculature as described herein. This quantification typically involves the setting of a threshold value to differentiate, for example, the pixels that represent active vasculature from static tissue within the angiogram. These 2D en face angiograms can be interpreted in a manner similar to traditional angiography techniques such as fluorescein angiography (FA) or indocyanine green (ICG) angiography, and are thus well-suited for clinical use. It is also common to generate en face images from structural OCT data in a manner analogous to that used to generate en face angiograms. Angiograms from different layers may also be color-coded and overlaid to present composite angiograms with encoded depth information; structural en face images may also be included in such composite image generation.

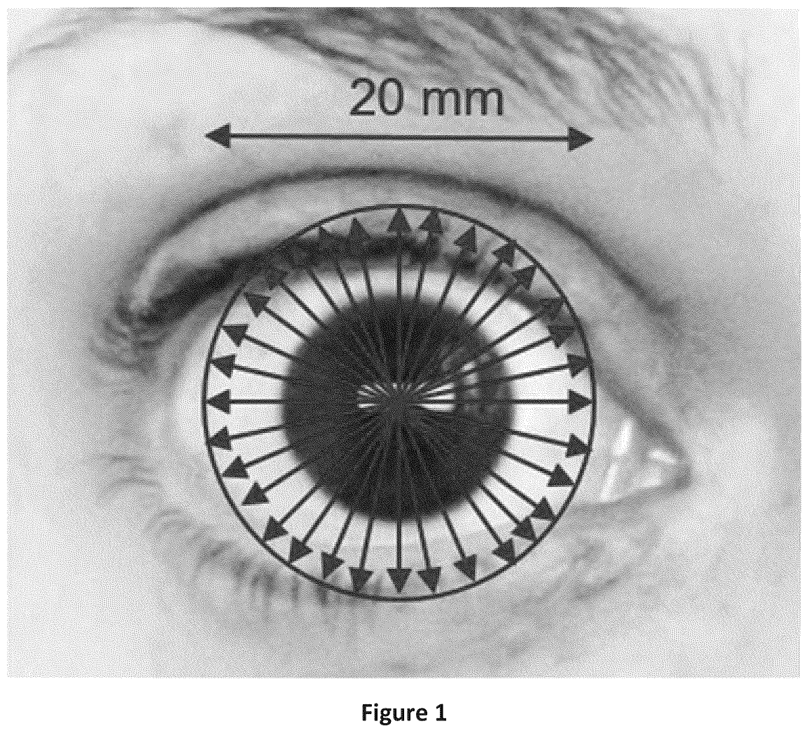

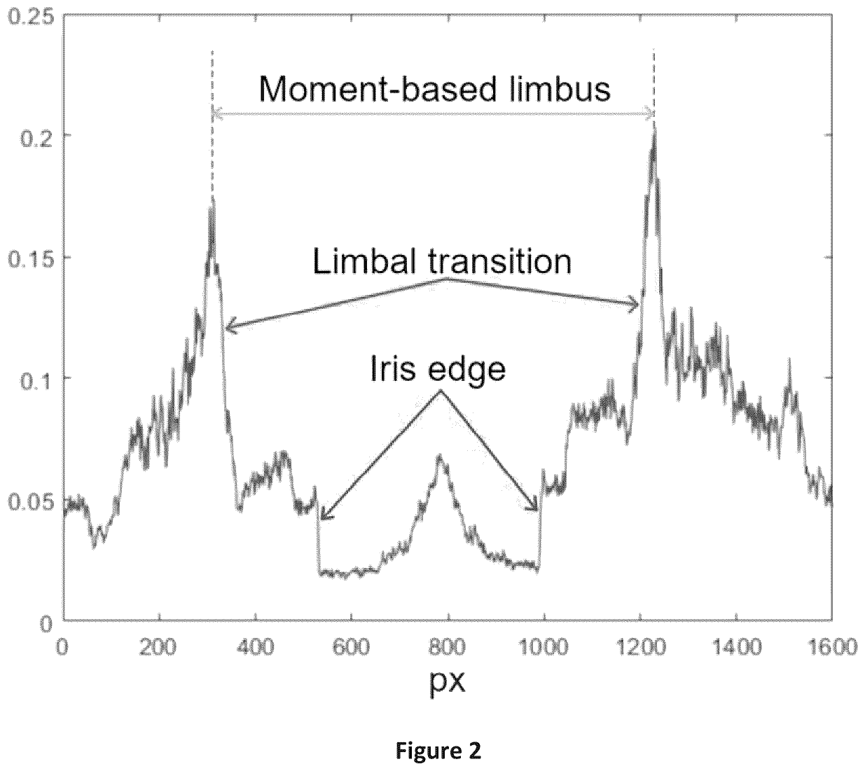

As briefly discussed above, embodiments herein provide a method to obtain an anterior ocular surface map (topography) with OCT. In some embodiments, the scan pattern comprises a series of radial lines intersecting at the approximate center of the cornea. In one example, at least 4 or 8 evenly spaced meridians are sampled. The lines are preferably at least 14-mm long so that both the corneal and scleral regions are covered. Alternative scan patterns, such as raster scans and/or OCT grid scans may be used. For example, raster scans of at least 14 mm×14 mm in size or other 3-dimensional volumetric scans centered on cornea and covering an area of at least 14 mm in diameter, may be used. These scans can be re-sampled to implement the topographic limbus detection. In one embodiment, 16 radial scans of 20-mm length are used (see FIG. 1). From each radial cross-sectional image (OCT B-frame), the second central moment of each axial scan (A-scan) is calculated and projected onto a radial profile representing the spatial variance of the OCT reflectance signal in the axial (depth) dimension. The limbus, as defined by the transition from clear cornea to white sclera, can be identified as positions of maximum gradient on this moment profile (FIG. 2). The pupillary boundary (iris edges) can also be located on this profile. While the example shown uses the second central moment, the zeroth, first, and/or third central moments of OCT A-scan signal can also be used to identify and locate these anatomic boundaries.

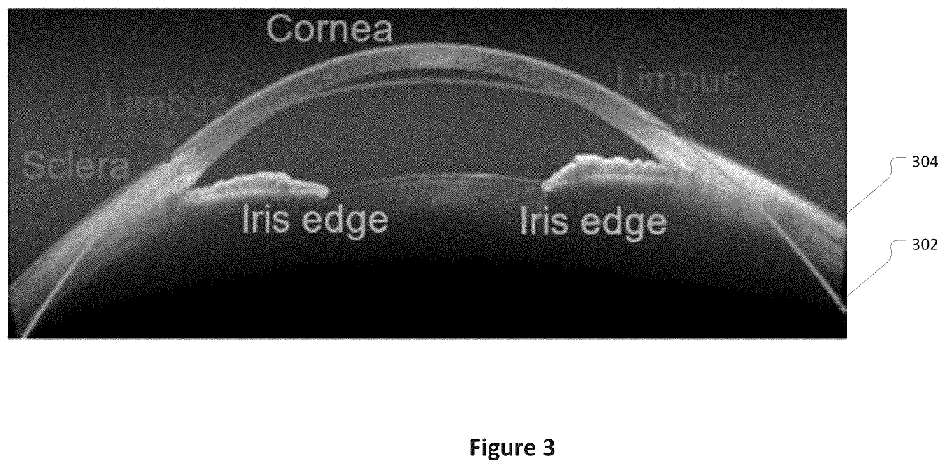

The moment-based limbus does not exactly match the external topographic limbus, which better correspond to where a scleral contact lens would settle around. To find the topographic limbus, on each B-frame, the ocular surface is separated into a central corneal region inside the moment-based limbus and a scleral region outside. Then the elevation profile of the external corneal surface is fitted with a 4^{th}-order polynomial, while the elevation profile of the external conjunctival surface over the sclera is fitted with a 2^{nd}-order polynomial. The intersection of the two best-fit surfaces defines the external topographical limbus (FIG. 3). The moment-based limbal locations and the topographic limbal locations are close to each other, but not identical.

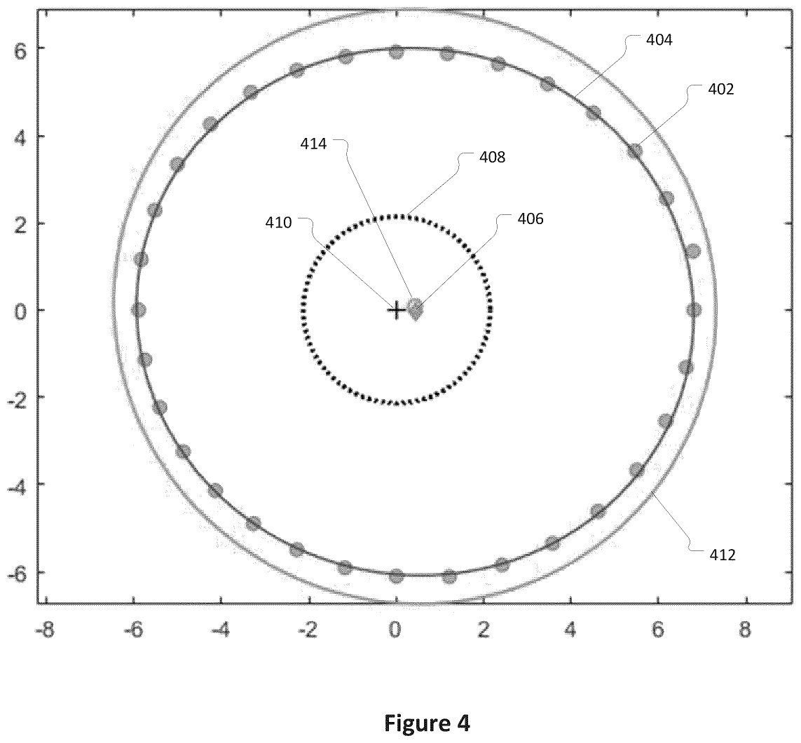

By combining the topographic limbal locations from all radial scans after registration, the 3-dimensional shape of the limbal ring is obtained (FIG. 4). The best-fit topographic limbal circle may be used to anchor the coordinate system for describing corneoscleral topography. The plane of the circle defines the x (horizontal) and y (vertical) axes with origin at the center of the circle. Elevation (z) is defined perpendicular to the circle plane.

In embodiments, the sinusoidal deviations of the topographic limbus from the best-fit limbal circle are decomposed using Fourier series. The second and third harmonic frequencies, respectively, are used to describe the ellipticity and ovality of the limbus. Double angle plots are used to visualize the orientation of the elliptical deviation. Similarly, Fourier analysis of the sinusoidal deviation of the topographic limbus elevation from the plane defined by the best-fit limbal circle defines the limbal toricity. For the enface representation the topographic limbus is fit with an ellipse. The pupil is fit with a circle along the iris edges identified on the radial scans (FIG. 4).

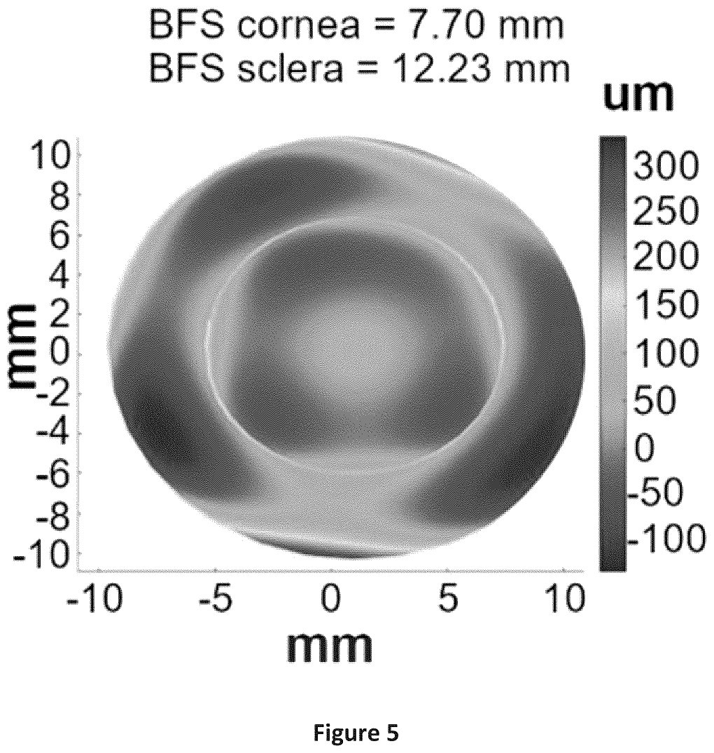

The 3D corneoscleral elevation surface may be generated after registering the surface profiles of all radial scans at the pupil center. In one example, the corneal surface inside the topographic limbal circle is fit with a sphere to obtain the average radius of curvature and to provide a reference for the mapping of relative float elevation. The corneal surface is also fit with an ellipsoid and the parameters of the ellipsoid define its steep and flat radii of curvature, toricity, and height. The conjunctival surface over the sclera outside the topographic limbal circle is fit with a sphere to obtain the average radius of curvature and to provide a reference to map float elevation. The conjunctival surface over the sclera is also fit with an ellipsoid and the parameters of the ellipsoid define its steep and flat radii of curvature and toricity. The surfaces can also be described in more detail by fitting with higher-order Zernike polynomial series (e.g. 8^{th} order). Zernike fitting is helpful as a method to reconstruct a complete topographic surface from sparsely sampled imaging data. It also smooths over small surfaces disturbances (e.g. dry spot). The corneal and conjunctival surfaces can be displayed as float elevation (relative to best-fit spheres as shown in FIG. 5), axial radius, and tangential radius maps according to standard convention.

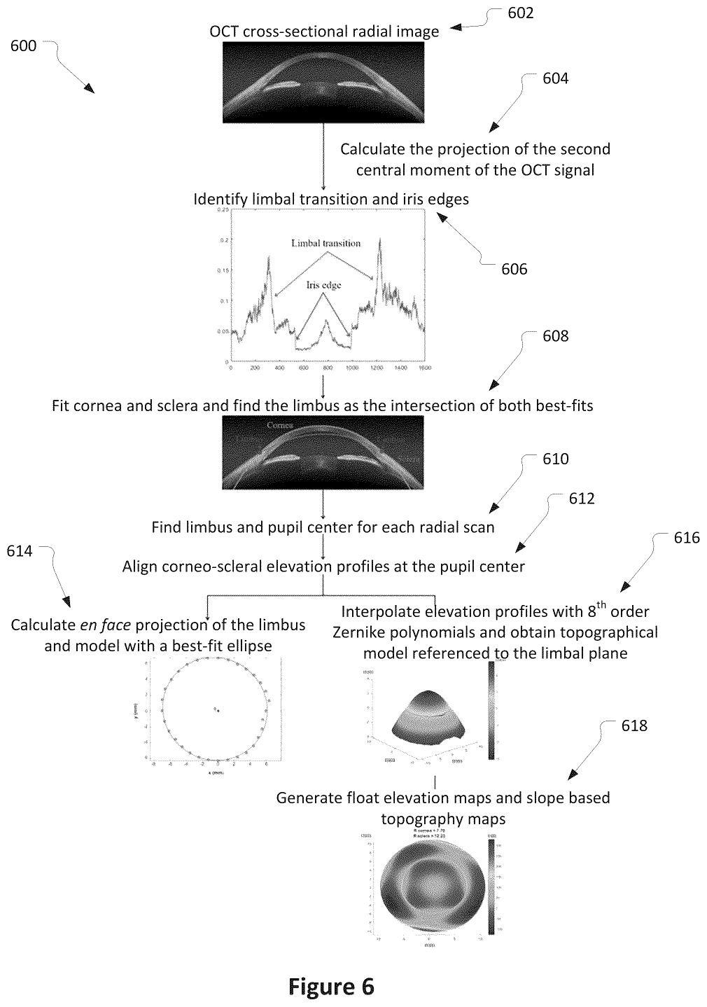

FIG. 6 illustrates an example process 600 to generate corneoscleral topography in accordance with various embodiments herein. The process 600 incudes, at 602, to obtain an OCT cross-sectional radial image. At 604, the process 600 includes to calculate the projection of the second central moment of the OCT signal. At 606, the process 600 further includes to identify limbal transition and iris edges (e.g., based on the projection). At 608, the process 600 further includes to fit the cornea and sclera and find the limbus as the intersection of both best-fits.

At 610, the process 600 includes to find the limbus and pupil center for each radial scan. At 612, the process 600 further includes to align corneo-scleral elevation profiles at the pupil center. At 614, the process 600 includes to calculate an en face projection of the limbus and model with a best-fit ellipse. At 616, the process 600 includes to interpolate elevation profiles with 8th order Zernike polynomials and obtain topographical model referenced to the limbal plane. At 618, the process 600 further includes to generate float elevation maps and slope based topography maps.

Although embodiments are generally described herein with reference to an OCT system, the method may also be applied to other anterior eye tomography devices and/or imaging techniques. For example, Scheimpflug imagins (e.g., using a rotating-slit Scheimpflug-camera based corneoscleral tomography device), ultrasound imaging, and/or another imaging technique may be used in some embodiments.

### EXAMPLES

The following examples are illustrative of the disclosed methods. In light of this disclosure, those skilled in the art will recognize that variations of these examples and other examples of the disclosed method would be possible without undue experimentation.

### Example 1

A study performed by the inventors is described further below. The study was performed using an ultrahigh-speed swept-source OCT prototype to scan the anterior eye. An automated algorithm was used to map the corneoscleral topography and determine 3-dimensional (3D) shape and size of the topographic limbus. The repeatability of the topographic limbal parameters was established and their correlations with traditional limbal measurements, such as white-to-white (WTW) and spur-to-spur diameters, were investigated.

A prospective cross-sectional observational study was performed to present a novel automatic method for 3-dimensional external limbal demarcation on corneoscleral topography derived from OCT. The limbal shape is investigated and compared to other limbal measures.

In the study, an ultrawide-field (20 mm) swept-source OCT was used to acquire anterior segment images. An automated algorithm was developed to obtain corneoscleral topography based on OCT radial scans on 16 meridians. The algorithm demarcates the topographic limbus based on the transition from corneal to scleral curvature radially. The internal limbal diameter is based on identification of the scleral spur on the OCT images by a human grader. White-to-white (WTW) diameter was obtained from the Pentacam HR Scheimpflug tomography system. The external topographic limbus was fit with a circle on a plane. Ellipticity was defined by the lateral limbal deviation from the best-fit circle. Toricity is defined by the axial deviation from the best-fit plane. Repeatability was assessed by the within-subject standard deviation from two repeated measurements.

In the study, 19 eyes from 12 subjects were analyzed. The topographic limbal diameter was 12.15±0.66 mm (mean±standard deviation) horizontally and 11.19±0.63 mm vertically. The internal and WTW horizontal limbal diameters were significantly smaller (11.71±0.27 mm and 11.84±0.28 respectively, generalized linear mixed-effects model (GLME), p<0.017). The vertical internal limbal diameter was significantly larger (12.06±0.44 mm, GLME p<0.05). The topographic limbus had significant ellipticity (0.25±0.13 mm, wider horizontally, repeatability of 0.07 mm) and toricity (0.25±0.14 mm, flatter horizontally, repeatability of 0.10 mm). Low Pearson correlations were found for the topographical limbus with the internal limbus (R²=0.023 and R²=0.0034, for horizontal and vertical diameters respectively) and with the WTW (R²=0.147 for the horizontal diameter).

The study shows that the proposed method to demarcate the 3D external topographical limbus is repeatable. The topographic limbal shape and size cannot be accurately derived from WTW nor internal limbus measures. This new technology may improve the process of scleral lens fitting.

Additional details of the study are described further below.

### Methods

### Study Subjects

Healthy volunteers were recruited at the Oregon Health & Science University Casey Eye Institute (Portland, Oregon, USA). All subjects were given written information of the nature, benefits, and risks of the study and signed an informed consent form. The study followed the tenets of the Declaration of Helsinki and the Health Insurance Portability and Accountability Act of 1996. The study protocol was approved by the Institutional Review Board prior to the start of the study. Volunteers with ocular pathologies, previous ocular surface surgery, previous ocular trauma or any medical condition that could affect the ocular surface topography were excluded from the study. Contact lens users were asked not to wear their contact lenses the day of the study.

### Optical Coherence Tomography Imaging

An ultrahigh-speed (325 kHz A-scan rate) ultrawide-field (20 mm field of view) 1310-nm wavelength swept-source OCT prototype was used to image the anterior segment of the eye. The imaging depth range was 15.9 mm (in air) with an axial resolution of 12.2 μm full-width-half-maximum (in air). The prototype used an electrically tunable liquid lens to dynamically change the focal plane position from the anterior cornea to the peripheral sclera and generate an extended range focused image of the entire corneoscleral surface. A customized scan pattern that optimized the corneoscleral coverage and focusing was designed consisting of 16 radial scans of 20 mm width. The scan pattern was repeated three times for each acquisition which is completed in 2.5 seconds. During the scan acquisition, subjects were asked to put the head on the chin and forehead rest and look at the internal fixation target. With a soft silicone-tipped rod, a second operator held the eyelids wide open during the acquisition. This was found to be necessary for adequate scleral exposure in most subjects. Both eyes of each subject were measured.

### Topographical Limbus and Internal Limbus Demarcation

Raw OCT data was exported and analyzed with a custom algorithm developed in the MATLAB® programming environment. The corneoscleral profile was corrected for the fan distortion in the OCT scan geometry. The approximate limbal locations were first established in each radial B-scan using the second central moment (variance) of OCT signal. The value of the moment is calculated from each A-scan and plotted as a function of lateral position along each OCT radial scan. Abrupt changes in the moment occur at the scleral and iris edges. These sharp transitions were used to obtain the pupil edges and approximate limbal positions.

The approximate limbal positions were used to separate the corneal and scleral regions in the OCT B-scans. The anterior surface profile of the corneal region was fitted with a 4th degree polynomial function and the scleral surface was fitted with a 2nd degree polynomial function. The intersection between these two fitted curves provided the location of the topographical limbus. The external topographical limbal points from the radial scans were combined to obtain the 3D limbal shape.

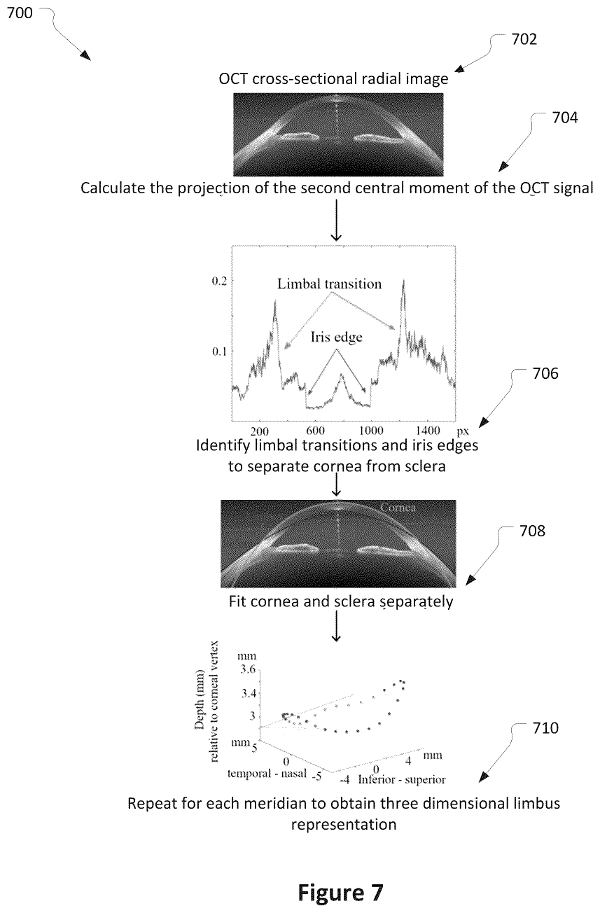

For example, FIG. 7 illustrates an example process 700 to obtain a three-dimensional limbus representation, in accordance with various embodiments. At 702, the process 700 includes to obtain an OCT cross-sectional radial image. At 704, the process 700 further includes to calculate the projection of the second central moment of the OCT signal. At 706, the process 700 includes to identify limbal transitions and iris edges. At 708, the process 700 includes to fit the cornea and sclera separately. At 710, the process 700 includes to repeat these operations for each meridian to obtain a three-dimensional limbus representation.

A similar procedure was used to obtain the 3D pupil boundaries. For each repetition of the OCT scan pattern, the radial scans were registered laterally at the pupil center and axially at the anterior corneal surface. Scans with significant eye movement could be detected by large displacement of the pupil center and removed from further analyses. The remaining valid repeats were used to obtain the average 3D limbus and pupil.

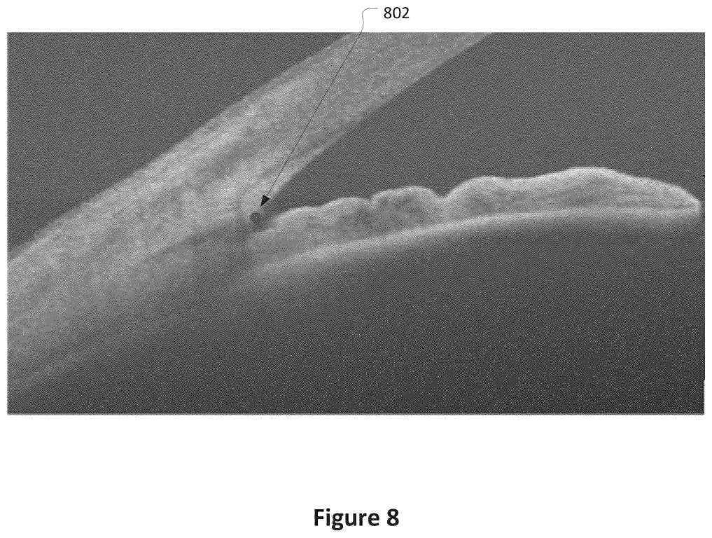

The internal limbus was defined by the position of the scleral spur on each B-scan manually located by a human grader. For this, the image was corrected for the distortion due to the change in the refractive index at the anterior ocular surface (dewarping). Then, the grader manually identified the scleral spur on each radial scan. The position of the internal limbus was averaged over the valid repeated scans. FIG. 8 shows an example of the internal limbus identification.

During the acquisition, is not always feasible to keep the eyelids out of the scan area, especially superiorly. Therefore, areas in the image obscured by the eyelids were automatically detected and excluded from further analysis. This can reduce the length of visible sclera on radial scans. If a radial scan has less than 2 mm of visible sclera on either side, both limbal points on the meridian were marked as undefined. To be considered valid, a corneoscleral topography scan must have no more than 8 missing limbal points (4 meridians) and never more than two adjacent missing points. Invalid scans were rejected from further analysis.

### Limbal Ellipticity, Ovality, and Toricity

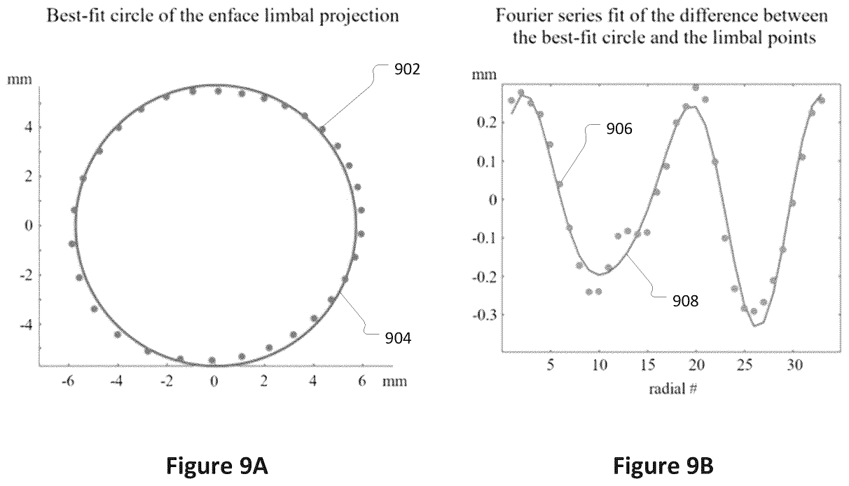

Limbal ellipticity and ovality are defined here as the deviation of the topographical limbal points from the best-fit circle on the limbal plane. The difference between the radial distance of the topographical limbus and the radius of the best-fit circle is fitted with a 3-term Fourier model (FIG. 9). The amplitude and orientation of the elliptical deviation is given by the sine and cosine terms of the second harmonic and the amplitude and orientation of the limbal ovality is given by the sine and cosine terms of the third harmonic.

The topographical limbus generally deviates from the best-fit plane in a saddle shape that implies toricity. Analogously to the previous analysis, the difference of the topographical limbus from the best-fit plane along the axial direction (anterior direction is positive) is fitted with a 2-term Fourier model where the amplitude and orientation of the limbal toricity is given by the sine and cosine terms of the second harmonic.

By this formulation, the distribution of the magnitude and orientation of the elliptical and toric deviation can be visualized using the double-angle plot similar to that used for astigmatism analysis. Analogously, the distribution of the magnitude and orientation of ovality can be visualized using the triple-angle plot. Left eyes were mirrored and pooled with right eyes for this analysis.

### Scheimpflug Imaging

For comparison with OCT results, the horizontal white-to-white (WTW) distance was obtained from a Pentacam HR (Oculus Optikgerate GmbH, software version 1.22 r09) Scheimpflug topography/tomography system.

### Statistical Analysis

Descriptive statistics are expressed as mean±standard deviation (SD). Data normality was tested by Shapiro-Wilk test. For paired and multiple comparisons, generalized linear mixed-effects models (GLME) were used to account for the correlation between left and right eyes within subjects. For all statistical testing, the significance level was set at p<0.05 and this significance level was adjusted with Bonferroni correction for multiple comparisons between the topographical limbus, internal limbus and WTW limbal diameter. Linear regression models and coefficients of determination R2, were used to determine the interconvertibility between the topographical limbus with the internal limbus and WTW diameters, ellipticity and toricity. A two-way intraclass correlation coefficient (ICC) was used to assess agreement between the three methods.

The repeatability of the limbal best-fit circle was calculated as the average of the within-subject standard deviation from two consecutive measurements (pooled standard deviation). For ellipticity, ovality and toricity the total vector repeatability was calculated as the square root sum of the squared sine and cosine components.

### Results

Fifteen subjects (30 eyes) were initially enrolled in the study (5 women and 10 men). Eleven scans were discarded due to poor eye opening, remaining 12 subjects (5 women and 7 men) and 19 eyes. The average age was 36±15 years. Out of those 19 eyes, 17 had enough corneal exposure to obtain all the limbal points, one scan was missing two limbal points and another four limbal points because the eyelids were not opened enough.

Table 1 shows the average values of the horizontal and vertical diameters for the topographical limbus, the internal limbus and the WTW. The horizontal diameter of the topographical limbus was significantly wider than the vertical diameter (GLME, p<0.0001) conversely to the internal limbus that was significantly wider along its vertical orientation (GLME, p=0.0003). These significant radial differences show that neither the topographical limbus nor the internal limbus should be assumed to be circular. Both the internal limbus and the WTW horizontal diameters were significantly smaller compared to the horizontal topographical limbal diameter (GLME p<0.001), and the internal limbus was significantly wider than the topographical limbus along the vertical meridian (GLME p<0.0001).

TABLE 1
Comparison of Limbal Diameters

| | Horizontal diameter | Vertical diameter |
|---|---|---|
| | (mm) | (mm) |
| Topographical limbus | 12.15 ± 0.66 | 11.19 ± 0.63 |
| Internal limbus | 11.71 ± 0.27 | 12.06 ± 0.44 |
| WTW | 11.84 ± 0.28 | — |
| p-value | <.001* | <.0001* |

WTW—white-to-white diameter measured with Pentacam.
*Statistically significant differences assessed with a generalized linear mixed effects model.

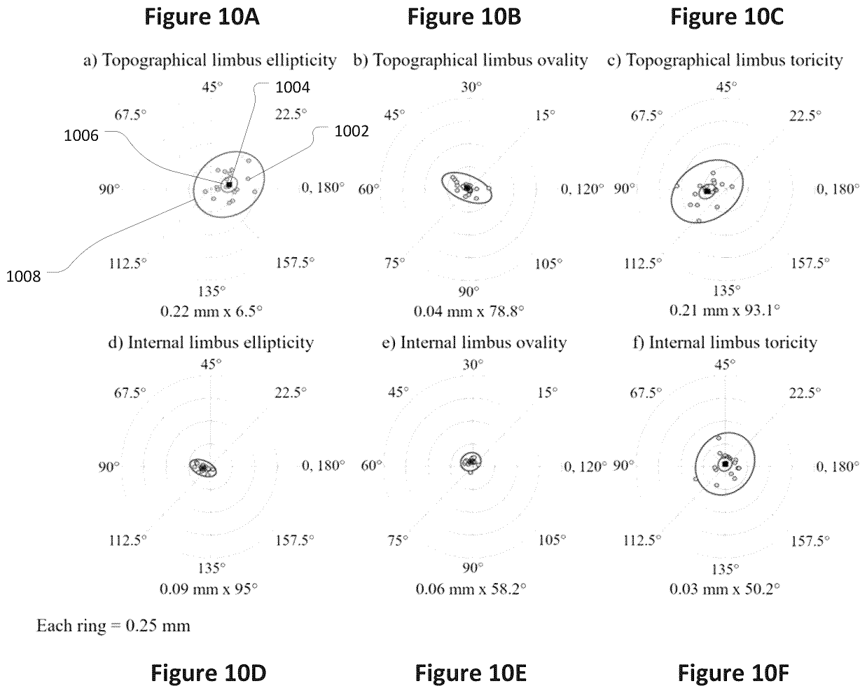

Table 2 shows the best-fit circle diameter together with the cosine and sine terms of ellipticity and ovality for the topographical and internal limbus. The topographical limbus was significantly more elliptical than the internal limbus as shown by the difference between the cosine terms (GLME p<0.001, Table 2). FIGS. 10A and 10D show the double angle plots of the magnitude and orientation of the elliptical deviation together with the average vectors calculated as the square root of the sum of the squared cosine and sine components The average vector for the ellipticity of the topographical limbus was 0.22 mm at 6.5° (wider horizontally) while for the internal limbus was 0.09 mm at 95° (wider vertically) defined according the right-eye convention orientation. The ovality magnitudes were small and the average ovality vector was not statistically different from zero for either the topographic or the internal limbus.

TABLE 2
Diameter, ellipticity, and ovality on the topographical and internal limbus

| | | Ellipticity (mm) | | Ovality (mm) | |
|---|---|---|---|---|---|
| | Diameter (mm) | Cosine term | Sine term | Cosine term | Sine term |
| Topographical limbus | 11.76 ± 0.61 | 0.22 ± 0.16 | 0.050 ± 0.13 | −0.033 ± 0.100 | 0.014 ± 0.061 |
| Repeatability | 0.054 | 0.060 | 0.037 | 0.030 | 0.028 |
| Internal limbus | 12.03 ± 0.35 | −0.087 ± 0.053 | −0.018 ± 0.034 | −0.025 ± 0.041 | 0.051 ± 0.037 |
| Repeatability | 0.021 | 0.040 | 0.107 | 0.053 | 0.044 |
| p-value | .0761 | <.001* | .0434 | .755 | .033 |

Diameter of the best fit circle and elliptical/oval deviations are shown as group average ± standard deviation.
Repeatability is assessed by pooled within-subject standard deviation.
*marks statistically significant differences between topographical and internal limbus assessed with a generalized mixed-effects model with Bonferroni correction

The toricity shows the saddle-shaped variations in the limbal elevation relative to the best-fit plane. This toricity is greater for the topographical limbus than for the internal limbus (GLME, p<0.001, Table 3). FIGS. 10C and 10F show the distribution of magnitude and orientation of limbal toricity for the topographical and internal limbus respectively. There was a statistically significant average toricity vector in the topographic limbal elevation at 93°—the limbal elevation is higher (more anterior) along the vertical meridian compared to the horizontal meridian. For the internal limbus, the average toricity vector was not significantly different from zero.

TABLE 3
Toricity on the topographical and internal limbus

Toricity (mm)

| | Cosine term | Sine term |
|---|---|---|
| Topographical limbus | −0.22 ± 0.14 | −0.023 ± 0.12 |
| Repeatability | 0.081 | 0.056 |
| Internal limbus | −0.0052 ± 0.11 | 0.029 ± 0.12 |
| Repeatability | 0.056 | 0.090 |
| p-value | <.001* | .210 |

Limbal toricity is shown as group average ± standard deviation.
Repeatability is assessed by pooled within-subject standard deviation.
*marks statistically significant differences between topographical and internal limbus assessed with a generalized mixed-effects model with Bonferroni correction

Repeatability was assessed in 10 eyes that had two repeated measures by means of within subject standard deviation. Table 2 shows the repeatability of the cosine and sine terms for limbal ellipticity and ovality. The repeatability of the total vectors was 0.07 mm and 0.04 mm for topographical limbus ellipticity and ovality respectively. For the internal limbus, the repeatability of ellipticity and ovality total vectors was 0.11 mm and 0.07 mm respectively. Table 3 shows the repeatability of the cosine and sine terms for limbal toricity. The repeatability of the total vector for the limbal toricity was 0.10 mm and 0.11 mm for topographical and internal limbus respectively.

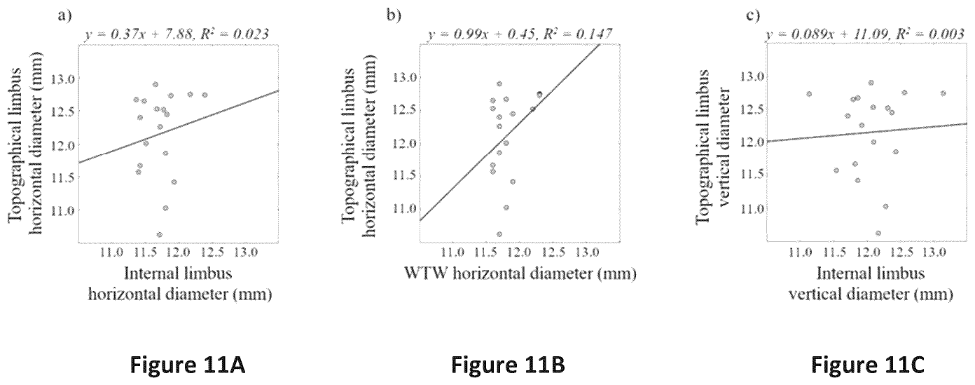

FIGS. 11A and 11B show the correlation of the topographical limbus with the internal limbus and WTW along the horizontal meridian. The coefficient of determination was R²=0.023 (p>0.05) for the linear regression of the horizontal topographical limbus vs horizontal internal limbus and R²=0.147 (p>0.05) for the linear regression of horizontal topographical limbus vs horizontal WTW. The ICC between the three methods along the horizontal meridian was 0.253. FIG. 11C shows the linear regression of the vertical topographical limbus vs vertical internal limbus, R²=0.0034 (p>0.05). The ICC between both methods was 0.223. The correlation coefficient between topographic and internal limbal ellipticity was R²=0.0033 (p>0.05) for the sine term R²=0.00013 (p>0.05) for the cosine term, and ICC=0.028 The correlation coefficient between topographic and internal limbal toricity was R²=0.003 (p>0.05) for the sine term, R²=0.037 (p>0.05) for the cosine term and ICC=0.497.

### DISCUSSION

The present inventors have developed an automatic method to demarcate the topographical limbus based on the shape of the anterior corneoscleral surface from OCT scans and described a new system to define limbal ellipticity and toricity. Additionally, the study investigated the structural differences between the topographical limbus and two other anatomical landmarks commonly used in the clinical practice: the WTW and the internal limbus defined as the spur-to-spur distance.

Precise limbus demarcation remains a challenge without consensus between different approaches on which is the most appropriate. This is partly because instead of being an abrupt boundary, the limbus is a transitional band both internally and externally. Different methods are used to measure the limbal diameter such as assessing the color transition from cornea to sclera, measuring the internal spur or angle recess diameters or looking to the topographical change of the corneoscleral profile. However, the significant differences and low correlation in size and shape between the topographical limbus and the WTW or the internal limbus show that they are not interchangeable nor interconvertible. Along the axial plane the significant differences the low correlation coefficients and the poor ICC between internal and topographical limbus show that they are not interchangeable. Which measure should be used will ultimately depend on the final purpose of the measure. Since a scleral lens sits on the ocular surface, that leads to accept the limbal characterization based on the topographical change of the anterior ocular surface as the most appropriate basis for scleral lens fitting rather than other landmarks.

The WTW distance estimates the limbal diameter based on the color change between iris and sclera. It is probably the most commonly used method to determine the limbus in the clinical practice because it is simple and quick to perform. When measured subjectively with calipers, accurate and reliable WTW demarcation can be ambiguous as the transition between the cornea and the sclera is seen as a color gradient transition instead of an abrupt change. The width of the limbal area is between 1 mm and 2 mm and it can be difficult to establish a precise point. Indeed, there is a wide range of WTW distances reported in the literature. Additionally, it is known that the WTW and the topographical limbus are not equivalent. Along the horizontal axes the WTW was significantly smaller than the topographical limbus but wider than the internal limbus.

Another landmark used to identify the limbal transition is the internal limbus either measured as the spur-to-spur or as the angle-to-angle diameters. Previous studies (see Skrok M K, Alonso-Caneiro D, Przeździecka-Dołyk J, Siedlecki D. Comparison of Subjective and Objective Methods of Corneoscleral Limbus Identification from Anterior Segment Optical Coherence Tomography Images. Optom Vis Sci 2021; 98:127-36. https://doi.org/10.1097/OPX.0000000000001637, hereinafter “Skrok et al.”) assessed the horizontal diameter of the topographical and internal limbus and found that the second was smaller. However, this study was restricted to the horizontal meridian. To properly describe the limbal shape it is important to do so along the different radial directions and not only along the horizontal. Since the limbus does not have radial symmetry, extrapolating the horizontal diameter to the other orientations would lead to inaccuracies that might have a posterior impact in scleral lens fitting and design. An advantage of the method described herein is that it provides a 360° characterization. The results showed that the width of the topographical limbus is significantly different from the internal limbus not only along the horizontal but also along the vertical meridian. While the external limbus was smaller vertically than horizontally, the internal limbus was larger vertically than horizontally. The low coefficient obtained for the correlation between internal and external limbus and the poor ICC show that the topographical limbal diameters cannot be derived from the internal limbal diameters and assuming that they are interchangeable would lead to inaccuracies.

Given that the limbus is not circular it becomes important to find a way to model its ellipticity. The techniques described herein may characterize the limbal asymmetry by fitting the deviation of the limbus from its best-fit circle with a 3-term Fourier function. This way, the ellipticity corresponds to the terms with angular frequency of two and the distribution of the amplitude and orientation of the elliptical component can be easily visualized using the double angle plots. Following this approach, the third frequency Fourier terms correspond to the limbal ovality and can be visualized using triple angle plots. This analysis can be also used to investigate the shape of the limbus along the axial direction (elevation) to describe its toricity. This analysis showed a good test-retest repeatability. The topographical limbus has been found to have a significant ellipticity being wider along the horizontal meridian, opposite to the internal limbus that has lower ellipticity and it is wider along the vertical meridian.

Less research has been done to describe the toricity of the topographical limbus. This is only possible with techniques that provide 3D data. The topographical limbus does not lie on a plane, but it has a saddle like shape being deeper on the nasal and temporal quadrants and shallower on the inferior and superior quadrants. The toricity of the limbus was consistent with its ellipticity. Since the transition between cornea and sclera occurs at a shorter radius for the vertical meridian compared to the horizontal meridian, it causes the limbus to remain more elevated (more anterior). The internal limbal toricity was smaller and not correlated with the topographical limbal toricity.

Most of the studies assessing the limbal structure use manual identification of the landmarks. Skrok et al. showed that despite there being a strong linear relationship between manual and automatic methods, there is a significant bias, with automatic methods giving larger values compared to the manual ones (around 0.20 mm difference). Additionally, worse repeatability has been found for manual methods showing the advantage of using a reliable automatic method. Accordingly, some efforts have been done to automate the external limbus demarcation using different imaging techniques. One approach has been to calculate the radius of curvature of the anterior surface of the eye and locate the limbus as the point where there is an abrupt radial change. Calculating the surface curvature is susceptible to local disturbances that are commonly found in the corneoscleral surface as pinguecula, conjunctivochalasis or pterygium. These methods also rely on precise segmentation of the anterior surface, which can be a problem with some instruments with limited depth of focus and prone to errors due to image artifacts. In addition, for patients with highly deformed corneas as keratoconic patients, edematous corneas or bullous keratopathy, this method might be inaccurate. Other techniques (see Consejo A, Iskander D R. Corneo-scleral limbus demarcation from 3D height data. Cont Lens Anterior Eye 2016; 39:450-7. https://doi.org/10.1016/j.clae.2016.05.001, hereinafter “Consejo et al.”) used an eye surface profilometer to obtain anterior segment elevation data which was fitted with a second radial order Zernike polynomial. The residual between the original elevation data and the fit was used to locate the limbus as the point where the curvature changes. Relaying on the differences between the original data and a single fit for all the corneoscleral surface can lead to misinterpretation in eye with extreme curvatures, irregular corneas or eyes with local disturbances. Additionally, a schema to deal with artifacts, eyelids and eye motion would need to be applied.

The techniques described herein will be more robust for those eyes with local irregularities as the initial transition between cornea and sclera is determined based on the change of the intensity variance that happens between different tissues being more robust to potential local disturbances around the limbus and less affected by defocus. Because the cornea and the sclera are fitted separately, the method may also be more reliable in eyes with abnormal radius of curvature or irregular corneas. In addition, the high speed of the OCT used in this study reduces motion artifacts and allows the acquisition of three sets of the scan pattern in approximately 0.8 seconds each one. That way if one of the sets is affected by motion artifacts, it can be discarded.

The techniques described herein may require a minimum of scleral exposure to have a reliable scleral fit. Some scans of the initially enrolled subjects had to be discarded due to poor scleral exposure resulting in a smaller sample size. Another possibility would be to take multiple scans with different gaze positions and generate a composite image maximizing the scleral coverage. This would increase the measurement time and would be more computationally expensive.

Accordingly, ultrawide-field anterior segment OCT is capable of providing images to evaluate the anterior corneoscleral shape and topographical limbal transition in three dimensions. The described method for limbus demarcation is repeatable and has shown that the anterior topographical limbal size and shape cannot be reliably derivable from the WTW or internal limbal measurements. The topographical limbal diameter is larger horizontally than vertically and the limbus shape has a significant amount of ellipticity and toricity that should be considered when fitting scleral lenses. The described method of defining the topographic limbus provides anchor points for a system of OCT-based corneoscleral topography, which in turn may enable a more objective and streamlined scleral lens fitting process and could enable more customized designs.

### Example 2

The methods described in the present disclosure may be implemented in an integrated system that is fully automated or assembled from different components that may require some manual intervention. In general, a system according to the present disclosure may comprise the components of a corneal topography measuring device capable of measuring and generating a corneal topography and an optical coherence tomography device, wherein both devices are capable of producing data in digital format or in a format that can be digitized, and a processing unit. The corneal topography measuring device may include, but not be limited to, Placido-ring topography, slit-scan corneal topography, Scheimpflug-camera corneal tomography, raster photogrammetry, optical coherence tomography, or any other suitable cornea measuring devices known in the art. The processing unit may be a personal computer, a workstation, an embedded processor, or any other suitable data processing device commonly known in the art.

In addition to being implemented in a system, the methods of the present disclosure may also be provided in the form of software encoded on a computer readable medium for distribution to end users. Example computer media may include, but not be limited to, floppy disks, CD-roms, DVDs, hard drive disks, flash memory cards, downloadable files on an internet accessible server, or any other computer readable media commonly known in the art.

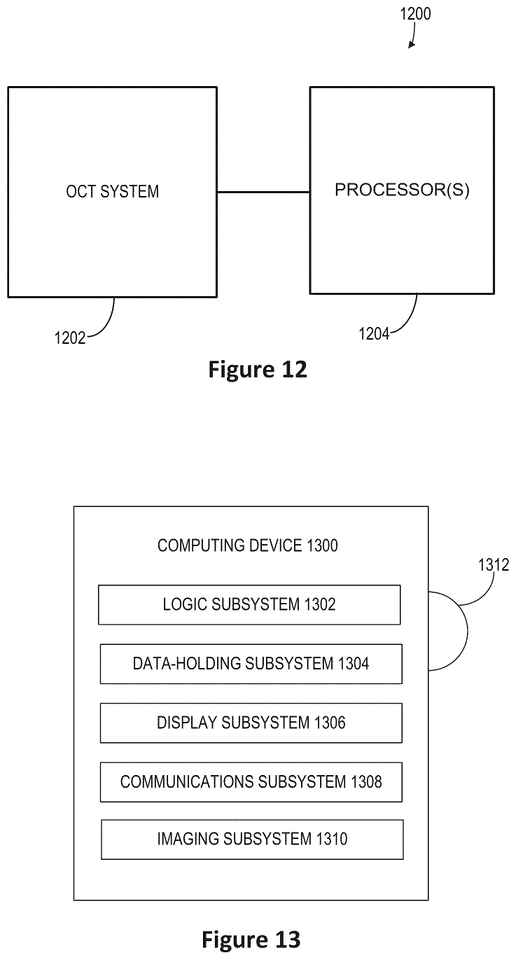

FIG. 12 schematically shows an example system 1200 for OCT image processing in accordance with various embodiments. System 1200 comprises an OCT system 1202 configured to acquire an OCT image comprising OCT interferograms and one or more processors or computing systems 1204 that are configured to implement the various processing routines described herein. OCT system 1200 can comprise an OCT system suitable for OCT angiography applications, e.g., a swept source OCT system or spectral domain OCT system.

In various embodiments, an OCT system can be adapted to allow an operator to perform various tasks. For example, an OCT system can be adapted to allow an operator to configure and/or launch various ones of the herein described methods. In some embodiments, an OCT system can be adapted to generate, or cause to be generated, reports of various information including, for example, reports of the results of scans run on a sample.

In embodiments of OCT systems comprising a display device, data and/or other information can be displayed for an operator. In embodiments, a display device can be adapted to receive an input (e.g., by a touch screen, actuation of an icon, manipulation of an input device such as a joystick or knob, etc.) and the input can, in some cases, be communicated (actively and/or passively) to one or more processors. In various embodiments, data and/or information can be displayed, and an operator can input information in response thereto.

In some embodiments, the above described methods and processes can be tied to a computing system, including one or more computers. In particular, the methods and processes described herein, e.g., the method depicted in FIG. 15 described below, can be implemented as a computer application, computer service, computer API, computer library, and/or other computer program product.

FIG. 13 schematically shows a non-limiting computing device 1300 that can perform one or more of the methods and processes described herein. For example, computing device 1300 can represent the processor 1204 included in system 1200 described above, and can be operatively coupled to, in communication with, or included in an OCT system or OCT image acquisition apparatus. Computing device 1300 is shown in simplified form. It is to be understood that virtually any computer architecture can be used without departing from the scope of this disclosure. In different embodiments, computing device 1300 can take the form of a microcomputer, an integrated computer circuit, printed circuit board (PCB), microchip, a mainframe computer, server computer, desktop computer, laptop computer, tablet computer, home entertainment computer, network computing device, mobile computing device, mobile communication device, gaming device, etc.

Computing device 1300 includes a logic subsystem 1302 and a data-holding subsystem 1304. Computing device 1300 can optionally include a display subsystem 1306, a communication subsystem 1308, an imaging subsystem 1310, and/or other components not shown in FIG. 13. Computing device 1300 can also optionally include user input devices such as manually actuated buttons, switches, keyboards, mice, game controllers, cameras, microphones, and/or touch screens, for example.

Logic subsystem 1302 can include one or more physical devices configured to execute one or more machine-readable instructions. For example, the logic subsystem can be configured to execute one or more instructions that are part of one or more applications, services, programs, routines, libraries, objects, components, data structures, or other logical constructs. Such instructions can be implemented to perform a task, implement a data type, transform the state of one or more devices, or otherwise arrive at a desired result.

The logic subsystem can include one or more processors that are configured to execute software instructions. For example, the one or more processors can comprise physical circuitry programmed to perform various acts described herein. Additionally or alternatively, the logic subsystem can include one or more hardware or firmware logic machines configured to execute hardware or firmware instructions. Processors of the logic subsystem can be single core or multicore, and the programs executed thereon can be configured for parallel or distributed processing. The logic subsystem can optionally include individual components that are distributed throughout two or more devices, which can be remotely located and/or configured for coordinated processing. One or more aspects of the logic subsystem can be virtualized and executed by remotely accessible networked computing devices configured in a cloud computing configuration.

Data-holding subsystem 1304 can include one or more physical, non-transitory, devices configured to hold data and/or instructions executable by the logic subsystem to implement the herein described methods and processes. When such methods and processes are implemented, the state of data-holding subsystem 1304 can be transformed (e.g., to hold different data).

Data-holding subsystem 1304 can include removable media and/or built-in devices. Data-holding subsystem 1304 can include optical memory devices (e.g., CD, DVD, HD-DVD, Blu-Ray Disc, etc.), semiconductor memory devices (e.g., RAM, EPROM, EEPROM, etc.) and/or magnetic memory devices (e.g., hard disk drive, floppy disk drive, tape drive, MRAM, etc.), among others. Data-holding subsystem 1304 can include devices with one or more of the following characteristics: volatile, nonvolatile, dynamic, static, read/write, read-only, random access, sequential access, location addressable, file addressable, and content addressable. In some embodiments, logic subsystem 1302 and data-holding subsystem 1304 can be integrated into one or more common devices, such as an application specific integrated circuit or a system on a chip.

FIG. 13 also shows an aspect of the data-holding subsystem in the form of removable computer-readable storage media 1312, which can be used to store and/or transfer data and/or instructions executable to implement the herein described methods and processes. Removable computer-readable storage media 1312 can take the form of CDs, DVDs, HD-DVDs, Blu-Ray Discs, EEPROMs, flash memory cards, USB storage devices, and/or floppy disks, among others.

When included, display subsystem 1306 can be used to present a visual representation of data held by data-holding subsystem 1304. As the herein described methods and processes change the data held by the data-holding subsystem, and thus transform the state of the data-holding subsystem, the state of display subsystem 1306 can likewise be transformed to visually represent changes in the underlying data. Display subsystem 1306 can include one or more display devices utilizing virtually any type of technology. Such display devices can be combined with logic subsystem 1302 and/or data-holding subsystem 1304 in a shared enclosure, or such display devices can be peripheral display devices.

When included, communication subsystem 1308 can be configured to communicatively couple computing device 1300 with one or more other computing devices. Communication subsystem 1308 can include wired and/or wireless communication devices compatible with one or more different communication protocols. As non-limiting examples, the communication subsystem can be configured for communication via a wireless telephone network, a wireless local area network, a wired local area network, a wireless wide area network, a wired wide area network, etc. In some embodiments, the communication subsystem can allow computing device 1300 to send and/or receive messages to and/or from other devices via a network such as the Internet.

When included, imaging subsystem 1310 can be used to acquire and/or process any suitable image data from various sensors or imaging devices in communication with computing device 1300. For example, imaging subsystem 1310 can be configured to acquire OCT image data, e.g., interferograms, as part of an OCT system, e.g., OCT system 1202 described above. Imaging subsystem 1310 can be combined with logic subsystem 1302 and/or data-holding subsystem 1304 in a shared enclosure, or such imaging subsystems can comprise periphery imaging devices. Data received from the imaging subsystem 1310 can be held by data-holding subsystem 1304 and/or removable computer-readable storage media 1312, for example.

It is to be understood that the configurations and/or approaches described herein are exemplary in nature, and that these specific embodiments or examples are not to be considered in a limiting sense, because numerous variations are possible. The specific routines or methods described herein can represent one or more of any number of processing strategies. As such, various acts illustrated can be performed in the sequence illustrated, in other sequences, in parallel, or in some cases omitted. Likewise, the order of the above-described processes can be changed.

The subject matter of the present disclosure includes all novel and nonobvious combinations and sub-combinations of the various processes, systems and configurations, and other features, functions, acts, and/or properties disclosed herein, as well as any and all equivalents thereof.

What is claimed is:

1. A computer-based method comprising:
obtaining an optical coherence tomography (OCT) data set for an OCT scan of a cornea and a sclera, wherein the OCT scan uses radial scan lines with a common center point; and
generating one or more topography maps based on the OCT data set.

2. The method of claim 1, wherein the center point corresponds to a center of the cornea.

3. The method of claim 1, wherein the radial scan lines include at least four meridians.

4. The method of claim 1, wherein the radial scan lines are at least 14 millimeters long.

5. The method of claim 1, wherein the OCT dataset includes axial scans along the radial scan lines, and wherein the method further comprises locating a limbal junction between the cornea and the sclera based on radial profiles of the central moments of the axial scans.

6. The method of claim 1, further comprising locating an external topographic limbus using curve fitting of a conjunctival surface in the sclera and a corneal surface in the cornea based on the one or more topographical maps.

7. The method of claim 1, further comprising generating a metric of limbal ellipticity and toricity using a decomposition of a topographic limbus deviation from a best-fit limbal circle.

8. The method of claim 7, wherein the decomposition is a Zernike decomposition.

9. The method of claim 1, wherein the one or more topographical maps include a conjunctival surface elevation map, and wherein the method further comprises generating supplemental data for the OCT dataset using polynomial fitting of the conjunctival surface elevation map in a region of the sclera.

10. The method of claim 1, further comprising displaying the one or more topographical maps or measuring one or more topographic parameters based on a coordinate system referenced to a best-fit topographic limbal circle.

11. A computer-based method comprising:
obtaining a three-dimensional (3D) volumetric scan of an eye, wherein the volumetric scan corresponds to a corneal region and a scleral region of the eye; and
locating a limbal junction or an external topographical limbus based on the 3D volumetric scan.

12. The method of claim 11, wherein the 3D volumetric scan is an optical coherence tomography (OCT) scan.

13. The method of claim 12, wherein the OCT scan uses radial scan lines.

14. The method of claim 11, wherein the 3D volumetric scan covers an area of at least 14 millimeters by 14 millimeters.

15. The method of claim 11, wherein the limbal junction is located based on central moments of axial scans of the 3D volumetric scan.

16. The method of claim 11, wherein the external topographic limbus is located using curve fitting of a conjunctival surface in the scleral region and a corneal surface in the corneal region.

17. A system comprising:
an optical coherence tomography (OCT) system to acquire an OCT dataset of an eye;
a logic subsystem; and
a data holding subsystem comprising non-transitory machine-readable instructions stored thereon that are executable by the logic subsystem to:
obtain, via the OCT system, an OCT data set for an OCT scan of a cornea and a sclera, wherein the OCT scan uses radial scan lines with a common center point; and
generate one or more topography maps based on the OCT data set.

18. The system of claim 17, wherein the center point corresponds to a center of the cornea.

19. The system of claim 17, wherein the radial scan lines include at least four meridians.

20. The system of claim 17, wherein the OCT dataset includes axial scans along the radial scan lines, and wherein the instructions are further executable by the logic subsystem to locate a limbal junction between the cornea and the sclera based on radial profiles of the central moments of the axial scans.

21. The system of claim 17, wherein the instructions are further executable by the logic subsystem to locate an external topographic limbus using curve fitting of a conjunctival surface in the sclera and a corneal surface in the cornea based on the one or more topographical maps.

22. The system of claim 17, wherein the instructions are further executable by the logic subsystem to generate a metric of limbal ellipticity and toricity using a decomposition of a topographic limbus deviation from a best-fit limbal circle.

23. The system of claim 17, wherein the one or more topographical maps include a conjunctival surface elevation map, and wherein the instructions are further executable by the logic subsystem to generate supplemental data for the OCT dataset using polynomial fitting of the conjunctival surface elevation map in a region of the sclera.

24. The system of claim 17, wherein the instructions are further executable by the logic subsystem to display the one or more topographical maps or measure one or more topographic parameters based on a coordinate system referenced to a best-fit topographic limbal circle.
